(19) United States
(12) Patent Application Publication
(10) Pub. No.: US 2026/0335412 A1
(43) Pub. Date: Oct. 29, 2026

(54) IMMUNOSUPPRESSIVE MEDICAMENT AND METHOD OF TREATMENT

(71) Applicant: RELAXERA PHARMAZEUTISCHE GESELLSCHAFT MBH & CO. KG, Bensheim (DE)

(72) Inventor: Thomas Bernd DSCHIETZIG, Bensheim (DE)

(73) Assignee: RELAXERA PHARMAZEUTISCHE GESELLSCHAFT MBH & CO. KG, Bensheim (DE)

(21) Appl. No.: 19/107,096

(22) PCT Filed: Aug. 30, 2023
(86) PCT No.: PCT/EP2023/073842

§ 371 Date: Feb. 27, 2025

(30) Foreign Application Priority Data
Aug. 31, 2022 (DE) 10 2022 121 976.3

(51) Int. Cl. A61K 38/22; A61P 29/00

(52) U.S. Cl. CPC A61K 38/2221; A61P 29/00

(57) ABSTRACT

Relaxin-2 for use as a broad-spectrum substitute of glucocorticoids, mineralocorticoids, corticosteroids and their ana-logues for treating tissue and endothelial injuries without incurring inducing gluconeogenesis, Cushing's syndrome and cushingoid adverse effects. Medicament and treatment for supporting the repair of tissue and endothelial injuries by a promotion of immune suppressive regulatory T cells (Treg), particularly, in case of autoimmune pathologies and organ transplantation.

### FIELD OF THE INVENTION

The present invention relates to medicinal preparations containing a low-molecular-weight peptide hormone of the human insulin superfamily and to medicinal preparations containing a Selective Glucocorticoid Receptor Modulator (SEGRM) dampening the innate immune system (A61K38/1754; A61K38/1751; A61K38/2221).

### BACKGROUND OF THE INVENTION

Glucocorticoids are steroid hormones secreted by the adrenal glands. They regulate various physiological functions and are important in maintaining basal and stress-related homeostasis. In pharmacologic doses, glucocorticoids and corticosteroids are effective immunosuppressants in treating numerous inflammatory, autoimmune, and lymphoproliferative diseases. At the cellular level, the glucocorticoids and corticosteroids are functionally mediated by the glucocorticoid receptor (GR), which, simply put, is the receptor to which cortisol, cortisone, and glucocorticoids bind. The GR belongs to a receptor superfamily of nuclear transactivating factors with over 200 members and is ubiquitously expressed in almost all human tissues and organs. The GR is a hormone-and ligand-dependent transcription factor and regulates or affects GR-responsive genes' expression, which probably accounts for 3% to 10% of the human genome. For example, the ligand-activated GR may upregulate the expression of anti-inflammatory proteins in the nucleus or inhibit the expression of pro-inflammatory proteins in the cytosol by preventing the translocation of other transcription factors from the cytosol to the nucleus. Inhibition may also occur by the ligand-activated GR complex binding to the same site on the DNA where another transcription factor would bind so that the other no longer has an effect. The functions of the activated GR complexes are pleiotropic and occur in various parts of the body: in the control of metabolism, body development, and immune response. This has led to steroids and glucocorticoids becoming the most used drugs (see for review Nicolaides N et al., Glucocorticoid Receptor in Feingold K R, Anawalt B, Boyce A, et al., eds. Endotext [Internet]. South Dartmouth (MA): 2021).

Relaxin was initially identified by its activity as a pregnancy hormone. Still, it not only has a function at the maternal-fetal interface (Hisaw FL in Experimental relaxation of the pubic ligament of the guinea pig, Proc. Soc. Exp. Biol. Med. 1926; 23:661-663). Relaxin is a heterodimeric peptide of about 6 kDa in which disulfide bridges like in insulin link an A chain and a B chain. The insulin superfamily includes insulin, insulin-like growth factors I and II, relaxin-1, -2, and -3; and insulin-like factors 3, 4, 5, and 6. In humans, three distinct forms of relaxin have been identified, of which relaxin-2 is the primary stored form and the only form secreted into the circulation. The biological role of relaxin-1 in humans is still unclear, as is that of relaxin-3, which is found only in the brain. Relaxin-2 has been shown to act as an endocrine and paracrine factor that dilates blood vessels and increases blood flow in tissues (see for review: Dschietzig T et al. in Relaxin: a pregnancy hormone as a central player of body fluid and circulation homeostasis, CMLS 2003; 60:688-700; Dschietzig T et al. in Relaxin-a pleiotropic hormone and its emerging role for experimental and clinical therapeutics, Pharmacol Ther 2006; 112:38e56).

(Numerous clinical applications of relaxin and relaxin agonists and antagonists have been proposed: for the treatment of cutaneous aging, androgenetic alopecia, atrophy, sclerosis, and miniaturization of the hair and hair follicles (EP0793505); for control of fetal growth (EP0991947), for increasing fertility (EP1473034), as an adjuvant in the differentiation of stem cells (EP1696948), for increasing arterial compliance (EP1765149), for diseases related to vasoconstriction (EP1854476), for tumor suppression (WO2007115414), for the treatment of diabetes and related complications (EP1909809), for treating multiple sclerosis and other neurodegenerative dysfunctions (EP2723366), for treating symptoms of aging and neurodegenerative dysfunctions (WO0048618), for treating glucotoxicity and impaired glucose tolerance (EP2817026), in treating dyspnea associated with acute heart failure (EP2829280), for treating heart failure with preserved ejection fraction (EP3145534), for treating CNS, CNS trauma, demyelinating disease and/or gliosis, multiple sclerosis (MS), Alzheimer's disease and Parkinson's disease, inflammatory conditions of CNS, diffuse cerebral sclerosis of Schilder; acute disseminated encephalomyelitis, acute hemorrhagic leukoencephalitis, transverse myelitis, and neuromyelitis optica, concussion, traumatic brain injury, shaken baby syndrome, traumatic spinal cord injury, traumatic brain injury, ionizing radiation, Korsakoff's syndrome, multiple systemic atrophy, prion disease, AIDS dementia complex, vasculitis, amyotrophic lateral sclerosis, Huntington's disease, autoimmune inflammatory disorders, retinal gliosis, encephalopathies, leukodystrophies, encephalitis, neuropathies (EP3347037), for treating various inflammatory conditions, acidic airway hyperreactivity, asthma, rheumatoid arthritis, gout, ankylosing spondylitis, inflammatory enteritis, myositis, systemic lupus erythematosus, sepsis, urticaria, psoriasis, allergic reactions (WO20220374669). However, a particular focus has been the use of relaxin for hemodynamic adaption and regulation of systemic vascular resistance in models of cardiac, renal, pulmonary, and hepatic infarction (WO9303755, WO0240500; Dschietzig T et al. in Plasma levels and myocardial expression of relaxin-2 are increased in human heart failure, CIRCULATION 2000, 102(18): 594; Coulson CC et al. in Central hemodynamic effects of recombinant human relaxin in the isolated, perfused rat heart model, Obstetrics & Gynecology 1996, 87(4): 610-612; Masini E. et al. in Relaxin counteracts myocardial damage induced by ischemia-reperfusion in isolated guinea pig hearts: evidence for an involvement of nitric oxide, Endocrinology 1997, 138:4713-4720; DiLascio G et al. in Cellular retrograde cardiomyoplasty and relaxin therapy for postischemic myocardial repair in a rat model, Tex Heart Inst J 2012, 39:488-499; Collino M et al. in Acute treatment with relaxin protects the kidney against ischemia-reperfusion injury., J Cell Mol Med 2013, 17:1494-1505; Bausys A et al. in Custodiol® supplemented with synthetic human relaxin decreases ischemia-reperfusion injury after porcine kidney transplantation, Int J Mol Sci. 2021, 22, 11417; Alexiou K et al. in Relaxin is a candidate drug for lung preservation: relaxin induced protection of rat lungs from ischemia-reperfusion injury, J Heart Lung Transplant 2010, 29:454-460; Teichmann SL et al. in Relaxin: a review of the biology and potential role in treating heart failure, Curr Heart Fail Rep 2010; 7:75-82). In addition, relaxin has been observed to reduce oxidative cell damages that occur in orthotopic kidney and liver transplants and liver perfusion systems (DE102005040492; Boehnert MU in Relaxin as an additional protective model of isolate perfused rat liver, Ann N Y Acad Sci 2005, 1041:434-440; Kageyama S et al. in Relaxin in Liver Transplantation: A Personal Perspective Mol Cell Endocrinol. 2019, 487:75-79; Jakubauskiene L et al. in Relaxin positively influences ischemia-reperfusion injury in solid organ transplantation: a comprehensive review, Int J Mol Sci. 2020, 21(2): 631ff). These findings appear to be consistent with observations that relaxin can act as a ligand of the GR in somatic cells, completely independent of the signaling cascades of the G-protein-coupled relaxin receptors RXFP1 and RXFP2 (formerly designated LGR7 and LGR8, respectively). Experiments in Hela cells have confirmed this, HEK cells and Th1-activated macrophages showing that relaxin-2 activates GR and that the relaxin-GR complex depresses stimulated secretion of the cytokines IL-1, IL-6, and TNF-α like dexamethasone (Dschietzig TB et al. in Identification of the pregnancy hormone relaxin as a glucocorticoid receptor agonist, FASEB J 2004, 18:1536-1538; Dschietzig T et al. in The pregnancy hormone relaxin binds to and activates the human glucocorticoid receptor, Ann N Y Acad Sci. 2005, 1041:256-71; Dschietzig T et al. in RXFP1-inactive relaxin activates human glucocorticoid receptor: further investigations into the relaxin-GR pathway, Regul Pept. 2009, 154:77-84; Dschietzig T et al. in Autoregulation of human relaxin-2 gene expression critically involves relaxin and glucocorticoid receptor binding to glucocorticoid response half-sites in the relaxin-2 promoter, Regul Pept. 2009, 155:163-73). The presence of relaxin also stimulates gene expression in the NOTCH1 intracellular domain (NICD), and the associated intercellular signaling appears to result in a diminished ischemia-reperfusion injury. However, ischemia-reperfusion injury (IRI) is an inevitable consequence of many clinical conditions, including trauma, sepsis, resection, and transplantation, and an immune-driven inflammatory response leading to cell death and early graft dysfunction. Liver biopsies in humans nevertheless suggest that the high NICD expression enhances resistance to IRI. The use of relaxin in organ preservation solutions for donor kidneys and livers appears promising to improve IRI resistance. On the other hand, their side effects limit the clinical use of glucocorticoids and GR-ligands.

Because almost all cells in the body express the same glucocorticoid receptor, the pharmacologically highly desirable effects of glucocorticoids and GR activation are equally associated with specific adverse effects caused thereby. Adverse metabolic and pharmacologic effects of prolonged glucocorticoid treatment include wound healing disorders, manifestation/deregulation of diabetes mellitus, adverse immunosuppression, increased risk of infection, osteoporosis, growth disturbances in children as well as myopathy/muscular atrophy, skin atrophy, steroid acne, hirsutism in addition to the typical symptoms of a Cushing's syndrome such as uncontrollable hypertension, disturbances of salt and water balance, psychological and neurological disorders, depression, etc. Therefore, an individual benefit-risk analysis must be performed for each glucocorticoid therapy, primarily when treating cancer patients and transplant patients who receive such hormone therapy. This applies to patients suffering from a hormone-refractory type of tumor who are to be treated by a combination of a specific antibody and steroids to inhibit the proliferation of cancer cells. This is particularly true for patients who have a chronic inflammatory disease but for whom the harmful side effects of prolonged glucocorticoid treatment would outweigh the painful or debilitating effects of the underlying disease. The known prior art provides no guidance as to whether and when treatment with relaxin is medically justified. The prior art in this field, therefore, represents a problem.

### SUMMARY OF THE INVENTION

The problem is solved by a pharmaceutical composition for treating a patient in need of a therapy dampening physiologic inflammatory reactions by the innate immune system, comprising an effective amount of synthetic human relaxin-2 and a pharmacological solvent, diluent, or excipient.

In some embodiments, the patient suspected of requiring a dampening of physiologic inflammatory reactions by the innate immune system is tested for one or more of the following serum parameters: serum HMGB1 (high-mobility group box protein) greater than or equal to 4 ng/ml; serum sTLR4 (soluble Toll-like receptor-4) greater than or equal to 0.5 ng/ml, serum sRAGE (soluble receptor of advanced glycation end-products) greater than or equal to 2 ng/ml, or serum calprotectin greater than or equal to 10 micrograms/ml.

The problem is further solved by a pharmaceutical composition for treating a patient suffering from inflammatory responses triggered by the innate immune system and/or requiring suppression through a ligand-activated glucocorticoid receptor, which comprises as an active ingredient an effective amount of synthetic human relaxin-2 and a pharmacological solvent, diluent, or excipient, whereby manifestation or deregulation of diabetes or the symptoms of a Cushing's syndrome are avoided.

In some embodiments, the patient suspected of having inflammatory responses triggered by the innate immune system has the following clinical criteria: pre-diabetes (HbA1C>5.7 and <6.5 %), obesity (BMI>30kg/m2), hypertension (stage 1 or higher according to the 2017 ACC/AHA Guidelines).

In some embodiments, the pharmaceutical composition comprising an effective amount of synthetic human relaxin-2 is for treating a patient in need of an altered body or organ development while also preventing manifestation or deregulation of diabetes or symptoms of a Cushing's syndrome. Said patient may already display have the following clinical characteristics: pre-diabetes (HbA1C>5.7 and <6.5 %), obesity (BMI>30kg/m2), hypertension (stage 1 or higher).

In some embodiments, the pharmaceutical composition comprising an effective amount of synthetic human relaxin-2 is for treating a patient having received an allotransplant and in need of a dampening of the innate immune system and inflammatory responses while preventing compromised wound healing, manifestation, or deregulation of diabetes or symptoms of a Cushing's syndrome. Said medical need of a dampening of the innate immune system is given in case of a transplant patient when one or more of the following four criteria are fulfilled: serum HMGB1 (high-mobility group box protein) greater or equal to 2 ng/ml, serum sTLR4 (soluble Toll-like receptor-4) greater or equal to 0.25 ng/ml, serum sRAGE (soluble receptor of advanced glycation end-products) greater or equal to 0.5 ng/ml, and/or serum calprotectin greater or equal to 4 micrograms/ml.

In some preferred embodiments, the pharmaceutical composition comprising an effective amount of synthetic human relaxin-2 is for treating a patient in need of chronic suppression of the innate immune system and inflammatory responses while preventing manifestation or deregulation of diabetes or symptoms of a Cushing's syndrome.

In some embodiments, said patient suspected of requiring a chronic suppression of inflammatory reactions manifests one or more medical criteria selected from serum HMGB1 (high-mobility group box protein) greater than or equal to 4 ng/ml, serum sTLR4 (soluble Toll-like receptor-4) greater than or equal to 0.5 ng/ml, serum sRAGE (soluble receptor of advanced glycation end-products) greater than or equal to 2 ng/ml, and/or serum calprotectin greater than or equal to 10 micrograms/ml.

In some other embodiments, said pharmaceutical composition is for treating a patient in need of hormone-refractory cancer therapy, including, but not limited to prostate cancer, breast cancer, or a primary cancer therapy through a ligand-activated GR, including, but not limited to Multiple Myeloma, Hodgkin's Disease, and other Lymphoid Cancers; Kaposi Sarcoma, the synthetic human relaxin-2 being used as supplement and substitute of the glucocorticoid receptor activating hormone.

Another aspect of the invention relates to a method of treating a patient which comprises testing said patient for one or more of the following clinical parameters: serum HMGB1 (high-mobility group box protein) greater than or equal to 4 ng/ml; serum sTLR4 (soluble Toll-like receptor-4) greater than or equal to 0.5 ng/ml; serum sRAGE (soluble receptor of advanced glycation end-products) greater than or equal to 2 ng/ml, and/or serum calprotectin greater than or equal to 10 micrograms/ml, and when given, administering to the patent an effective amount of synthetic human relaxin-2 within a pharmacological solvent, diluent, or excipient to dampen or suppress physiologic inflammatory reactions by the innate immune system.

In some embodiments, said method steps are used for treating a patient suffering from inflammatory responses triggered by the innate immune system and/or requiring suppression through a ligand-activated glucocorticoid receptor, wherein the active pharmacological ingredient is synthetic human relaxin-2 to avoid a manifestation or deregulation of diabetes or the symptoms of a Cushing's syndrome.

In some other embodiments, said method of treatment comprises administering an effective amount of synthetic human relaxin-2 a patient in need of an altered body or organ development while preventing manifestation or deregulation of diabetes or symptoms of a Cushing's syndrome.

In some embodiments, the method comprises administering an effective amount of synthetic human relaxin-2 to a patient who has received an allograft and requires suppression of innate immune and inflammatory responses without compromising wound healing, manifesting or deregulating diabetes, or inducing symptoms of a Cushing's syndrome, after testing the transplant patient positive on one or more of the following medical criteria: serum HMGB1 (high-mobility group box protein) greater or equal to 2 ng/ml, serum sTLR4 (soluble Toll-like receptor-4) greater or equal to 0.25 ng/ml, sRAGE (soluble receptor of advanced glycation end-products) greater or equal to 0.5 ng/ml, and/or serum calprotectin greater or equal to 4 micrograms/ml.

In some other embodiments, the method of treatment comprises administering an effective amount of synthetic human relaxin-2 to a patient diagnosed as needing hormone-refractory cancer therapy, including, but not limited to prostate cancer, breast cancer, or a primary cancer therapy by a ligand-activated GR, including, but not limited to Multiple Myeloma, Hodgkin's Disease, and other Lymphoid Cancers, Kaposi's sarcoma, wherein the synthetic human relaxin-2 is used to supplement and/or replace glucocorticoid receptor-activating hormone. Alternatively, in some embodiments, the method comprises administering an effective amount of synthetic human relaxin-2 to a patient who requires an extra or substitute for a glucocorticoid receptor activating hormone.

In some embodiments, the method of treatment comprises administering an effective amount of synthetic human relaxin-2 to a patient diagnosed as needing immunosuppressive therapy, wherein the synthetic relaxin-2 is used to supplement and/or replace glucocorticoid receptor-activating hormone to prevent or avoid a manifestation or deregulation of diabetes or symptoms of Cushing's syndrome, mainly when the patient displays one or more of the following clinical features: pre-diabetes (HbA1C>5.7 and <6.5 %), obesity (BMI>30 kg/m2), hypertension (Level 1 or higher according to the 2017 ACC/AHA guidelines).

In some embodiments of the method of treatment, the patient is initially diagnosed as requiring chronic doses of corticosteroids and/or glucocorticoids and it comprises subcutaneously administering an appropriate amount of synthetic human relaxin-2 thereby avoiding a manifestation or deregulation of diabetes, wound healing disorders, or the symptoms of Cushing's syndrome.

In some embodiments of the method of treatment described above, the patient suffers from forms of autoimmune or rheumatic diseases; ankylosing spondylitis (AS) and spondylarthrites, fibromyalgia, gout, infectious arthritis, lupus, systemic autoimmune disease, osteoarthritis (OA), psoriatic arthritis (PsA) and inflammatory types of arthritis, rheumatoid arthritis (RA).

In some embodiments of the method of treatment described above, the method comprises testing the patient for one or more of the following clinical parameters: serum HMGB1 (high-mobility group box protein) greater than or equal to 4 ng/ml; serum sTLR4 (soluble Toll-like receptor-4) greater than or equal to 0.5 ng/ml, serum sRAGE (soluble receptor of advanced glycation end-products) greater than or equal to 2 ng/ml, and/or serum calprotectin greater than or equal to 10 micrograms/ml, and when given, administering to the patent an effective amount of synthetic human relaxin-2 within a pharmacological solvent, diluent, or excipient to dampen or suppress physiologic inflammatory reactions by the innate immune system.

In some embodiments of the method of treatment described above, the method comprises treating a patient suffering from SIRS (systemic inflammatory response syndrome), autoimmune or rheumatic diseases, thyroiditis, gastritis, insulitis, sialoadenitis, adrenalitis, oophoritis, glomerulonephritis, polyarthritis, ankylosing spondylitis (AS) and spondylarthrites, fibromyalgia, gout, infectious arthritis, lupus, systemic autoimmune disease, osteoarthritis (OA), psoriatic arthritis (PsA) and inflammatory types of arthritis, rheumatoid arthritis (RA), SARS-Covid 19 und SARS.

In some embodiments of the method of treatment described above, the method comprises treating a patient suffering from immunological reactions by the innate immune system and/or displays the following clinical criteria: pre-diabetes (HbA1C>5.7 and <6.5 %), obesity (BMI>30 kg/m2), hypertension (stage 1 or higher according to the 2017 ACC/AHA Guidelines).

In some embodiments of the method of treatment described above, the method comprises treating a patient suffering from inflammatory responses triggered by the innate immune system and/or requires suppression of inflammatory responses through a ligand-activated glucocorticoid receptor, wherein the active pharmacological ingredient is synthetic human relaxin-2 to avoid a manifestation or deregulation of diabetes, wound-healing disturbance, and/or the symptoms of Cushing's syndrome.

In some embodiments of the method of treatment described above, the method comprises treating a patient who has received an allograft and requires suppression of the innate immune system and inflammatory responses, after testing the patient positive on one or more of the following medical criteria: serum HMGB1 (high-mobility group box protein) greater or equal to 2 ng/ml, serum sTLR4 (soluble Toll-like receptor-4) greater or equal to 0.25 ng/ml, serum sRAGE (soluble receptor of advanced glycation end-products) greater or equal to 0.5 ng/ml, and/or serum calprotectin greater or equal to 4 micrograms/ml.

In some embodiments of the method of treatment described above, the method comprises treating a patient who is in need of hormone-refractory cancer therapy, including, but not limited to prostate cancer, breast cancer, or a primary cancer therapy by a ligand-activated GR, including, but not limited to Multiple Myeloma, Hodgkin's Disease, and other Lymphoid Cancers, Kaposi's sarcoma, wherein synthetic human relaxin-2 is used to supplement and/or replace glucocorticoid receptor-activating hormone.

In some embodiments of the method of treatment described above, the method comprises treating a patient diagnosed as requiring immunosuppressive therapy, wherein synthetic relaxin-2 is used to supplement and/or replace glucocorticoid receptor-activating hormone to prevent or avoid a manifestation or deregulation of diabetes or symptoms of Cushing's syndrome, particularly when the patient displays one or more of the following clinical features: pre-diabetes (HbA1C>5.7 and <6.5 %), obesity (BMI>30 kg/m2), hypertension (Level 1 or higher according to the 2017 ACC/AHA guidelines).

Further aspects and advantages of embodiments of the present invention will become apparent from the following detailed description, taken in conjunction with the accompanying drawings, illustrated by way of example of the principles of the invention.

### BRIEF DESCRIPTION OF THE DRAWINGS

In the figures and drawings appended hereto:

FIG. 1 is a diagram showing a microscale thermophoresis of the high-affinity binding (K_{D}~5 nM) of H2 relaxin to helix 12 of the ligand-binding domain of the human GC receptor in the absence of the co-activator TIF2.

FIG. 2 is a diagram of a microscale thermophoresis showing the lower affinity interaction (K_{D}~500 nM) of H2 relaxin to the ligand-binding domain of the human GC receptor.

FIG. 3 is a data plot of the release of lactate dehydrogenase (LDH) from cultured primary mouse hepatocytes after induced cell injury: Control (no cell injury); induction by H₂O₂; induction by H₂O₂ after treatment of cells with relaxin-2 (Rlx) or dexamethasone (Dx); after knockdown of the GC receptor by added siRNA (GRsi) and scrambled siRNA (scr) as a control-all data in percent of detergent-induced maximal cytotoxicity.

FIG. 4 is a data plot of the release of cleaved caspase-3 from cultured primary mouse hepatocytes after induced cell injury: Control (no cell injury); cell injury by H₂O₂; after treatment of cells with relaxin-2 (Rlx) or dexamethasone (Dx); after knockdown of the GC receptor by addition of siRNA (GRsi) and scrambled siRNA (scr) as a control-all data normalized to β-actin and adjusted to the effect of H₂O₂.

FIG. 5 is a data plot comparing the relative amounts of cytosolic GC receptor (GR) in cultured primary mouse hepatocytes after induced cell injury: Control (no cell injury), by addition of H₂O₂; after treatment of cells with relaxin-2 (Rlx) or dexamethasone (Dx); relaxin-2 or dexamethasone alone-all data normalized to control.

FIG. 6 is a data plot comparing the relative amounts of mitochondrial pyruvate dehydrogenase lipoamide kinase isoenzyme 4 (PDK-4) in cultured primary mouse hepatocytes after induced cell injury: Control (no cell injury), cell injury by addition of H₂O₂; cell injury after treatment of cells with relaxin-2 (Rlx) or dexamethasone (Dx); added relaxin-2 or dexamethasone alone-all data normalized to control.

FIG. 7 is a data plot comparing concentrations of tumor necrose factor-alpha (pg/mL)—an adipokine and cytokine-in the supernatants of activated Th1-macrophages: Control (no activation); activation by lipopolysaccharides (endotoxin); after treatment of macrophages with relaxin-2 (Rlx) or dexamethasone (Dx) or Mifepristone-RU486 (RU) or LPS+RIx+RU486 or LPS+Dx+RU486.

FIG. 8 is a data plot comparing concentrations of interleukin-6 (pg/mL)—pro-inflammatory cytokine-in the supernatants of activated Th1-macrophages: Control (no activation); activation by lipopolysaccharides (endotoxin); after treatment of macrophages with relaxin-2 (Rlx) or dexamethasone (Dx) or Mifepristone-RU486 (RU) or LPS+Rlx+RU486 or LPS+Dx+RU486.

FIG. 9 is a data plot comparing concentrations of circulating tumor necrose factor alpha (pg/mL) in blood from rats 24 hours after challenge with E. coli endotoxin: Control (placebo-no endotoxin); challenge with E. coli endotoxin (125 μg LPS/kg body weight); 2 hours after continuous sc. infusion (4 ug/h) of synthetic relaxin-2 (Relaxera Pharmazeutische GmbH, DE); i.m. injection of dexamethasone (10 mg/kg); oral RU-486 (single dose of 10 mg/kg body weight) or combinations thereof;

FIG. 10 is a data plot comparing concentrations of fasting blood sugar levels (24 hours) in rats after exposure to E. coli endotoxin: control (placebo-no endotoxin); challenge with E. coli endotoxin (125 μg LPS/kg body weight); challenge 2 hours after continuous subcutaneous infusion (4 μg/h) of synthetic relaxin-2 (Relaxera Pharmazeutische GmbH, Bensheim, DE); intramuscular injection of dexamethasone (10 mg/kg im.; oral RU-486 (single dose of 10 mg/kg body weight) or combinations thereof.

FIG. 11 is a data plot comparing concentrations of fasting blood sugar levels (48 hours) in blood from rats after challenge with E. coli endotoxin: Control (placebo—no endotoxin); challenge with E. coli endotoxin (125 μg LPS/kg body weight); challenge 2 hours after continuous sc. infusion (4 μg/h) of synthetic relaxin-2 (Relaxera, Bensheim, DE); intramuscular injection of dexamethasone (10 mg/kg); oral RU-486 (single dose of 10 mg/kg body weight) or combinations thereof.

FIG. 12 is a data plot comparing the percentage of Treg macrophages (CD4+CD25+ regulatory T cells) in blood from rats after continuous sc. infusion (4 μg/h) of relaxin-2 or oral RU-486 (single dose of 10 mg/kg body weight) or both—all data as a percentage of the total white blood cell count (WBC).

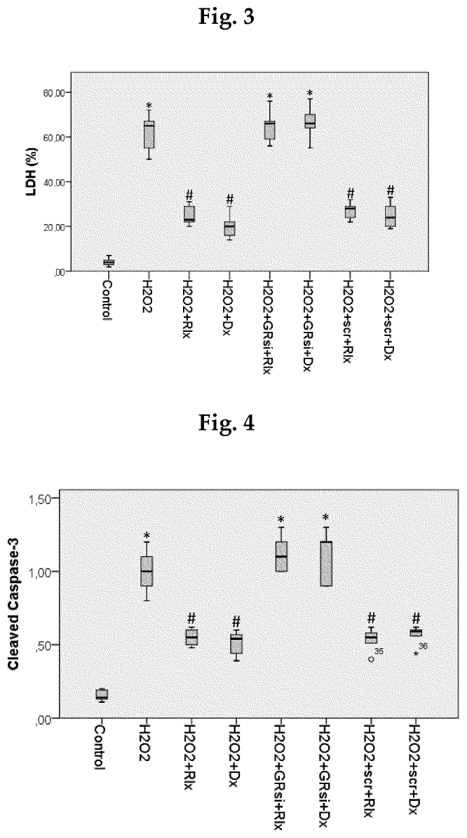

In FIGS. 3 to 12, the boxes indicate the interquartile range, with the inner line representing the median; the whiskers indicate 1.5-times the interquartile range; values above 1.5 times (outliers) and 3 times the interquartile range (extreme range) are shown as circles and stars, respectively.

### DETAILED DESCRIPTION OF THE INVENTION

Millions of patients take glucocorticoids to treat autoimmune and rheumatoid diseases, neurological disorders, pulmonary diseases, cancer, and other diseases and causes. However, the chronic side effects of glucocorticoids and their adverse consequences are much feared, particularly the negative impact due to glucocorticoid receptor (GR) down-regulation, steroid-induced hyperglycemia, or activation of gluconeogenesis as well as Cushing's syndrome. The present inventors have discovered that relaxin-2 binds the ligand-binding domain of the GR like steroids and glucocorticoids, forming an activated GR-ligand complex. The relaxin-GR complex activates the transcription of genes dampening the innate immune system but no genes activating gluconeogenesis, unlike the glucocorticoids or corticosteroids. This discovery enlarges the therapeutic applications of relaxin-2 for patients in need of a dampened innate immune system. This group of patients comprises for example patients who receive or have received an allotransplant, and cancer patients. Another large group are patients suffering from forms of tissue/endothelial injury or tissue-damaging diseases, including autoimmune or rheumatic tissue-damaging diseases comprising ankylosing spondylitis (AS) and spondylarthrites, fibromyalgia, gout, infectious arthritis, lupus, systemic autoimmune disease, osteoarthritis (OA), psoriatic arthritis (PsA), and inflammatory types of arthritis, rheumatoid arthritis (RA). Medications for these diseases include corticosteroids, oral and topical analgesics, non-steroidal anti-inflammatory drugs such as ibuprofen and COX-2 inhibitors, and disease-specific biologics. Tissue injury, endothelial injury, and endothelial cell activation, particularly in allograft rejection, can be detected and monitored by increased concentrations of calprotectin and/or S100A12 in extracellular fluids and the bloodstream. This is because endothelial cells also play a critical role in immune cell recruitment and extravasation. The calcium-binding S100 proteins, particularly calprotectin and S100A12, have a wide range of intracellular and extracellular functions, including regulation of calcium balance, cell apoptosis, cell migration, differentiation, proliferation, energy metabolism, and inflammation. The calcium-binding S100 proteins are released from the cytoplasm of the endothelial cell when triggered by tissue/cell damage, antibody stress, and endothelial stress. The S100 proteins then serve as danger signals, DAMP (damage-associated molecular pattern) molecules, and are involved in the regulation of immune homeostasis (macrophage migration, invasion, and differentiation), post-traumatic injury, and inflammation. They are therefore biomarkers in some specific diseases such as IBD (inflammatory bowel disease), although their multiple functions must be assigned to cell migration, differentiation, tissue repair, immune homeostasis, and inflammation management. The lack of commonly available diagnostic tests for tissue injury, endothelial injury, endothelial stress, and anti-endothelial cell antibody binding, which undoubtedly lead to allograft dysfunction and allograft rejection, makes calcium-binding S100A12 and calprotectin biomarkers for endothelial activation, immune cell recruitment, endothelial injury, endothelial antibody binding, and complement activation.

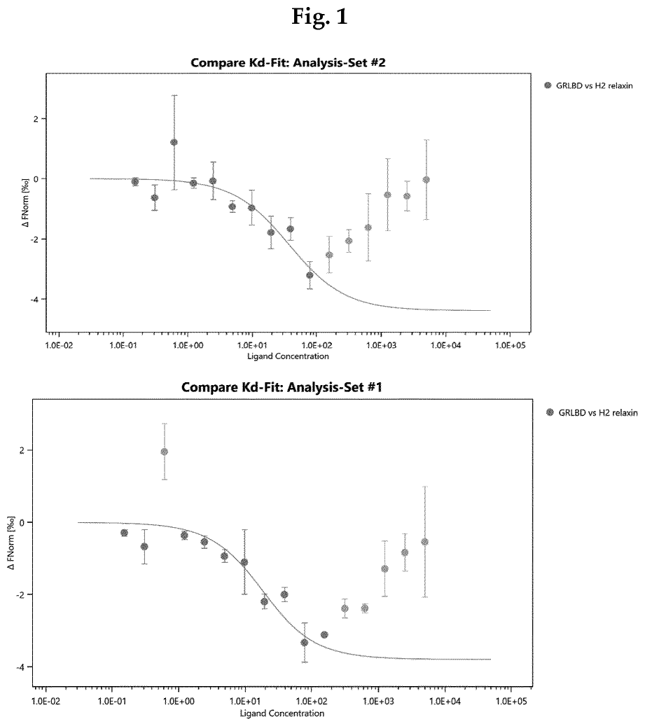

Furthermore, the present inventors discovered that relaxin-2 not only binds to the glucocorticoid receptor as shown in FIG. 1 but that the experimental results in FIG. 12 further indicate that the administration of relaxin-2 leads to a specific activation and promotion of regulatory T cells at local and systemic levels, likely induced by the relaxin-GR complex. This allows suppression of immune responses and significantly widens the use of relaxin-2 as an active ingredient in pharmaceuticals for treating abnormal, excessive, and undesired tissue-damaging immune responses to self-and foreign antigens. While the promotion of peripherally induced Treg cells could also be achieved by the administration of glucocorticoids, such treatment is disadvantageous due to the cushingoid adverse effects of glucocorticoids, corticosteroids, and synthetic analogs thereof. Their adverse effects are well-known and numerous (cf. 2022ICD-10 CM Code T38.0X5A).

Regulatory T cells (Treg cells) were originally defined as CD4+ T cells with a high expression of CD25 (interleukin-2 receptor α-chain). The regulatory T cells are further classified into thymic and peripherally induced Treg cells based on where they develop. The Foxp3 gene, a member of the Forkhead/winged-helix family of transcriptional regulators, was discovered to be an important regulator in the development of Treg cells based on the following findings: Scurfy mice with a frameshift mutation in the Foxp3 gene have T cell inflammation in multiple organs and a lethal autoimmune disease due to effector T cell activation and increased cytokine production caused by the absence of Treg cells. In addition, mutation of the Foxp3 gene in humans leads to IPEX syndrome (X-linked immune dysregulation, polyendocrinopathy, and enteropathy). In addition, forced expression of FoxP3 in naive T cells leads to immune suppressive function. CD4/CD25-naive T cells transfected with the Foxp3 gene can transform into CD4⁺CD25⁺ T_{reg} -like cells that produce inhibitory cytokines and express typical T_{reg} -cell molecules such as CD25, cytotoxic T-lymphocyte antigen-4 (CTLA-4), and glucocorticoid-induced tumor necrosis factor (TNF) receptor-related protein (GITR). Thus, FoxP3 is a lineage-specific marker and an important regulatory gene for the generation, maintenance, and immune suppressive functions of Treg cells. Regulatory T cells are required to suppress abnormal or excessive immune responses and to maintain homeostasis and self-tolerance by inhibiting T cell proliferation and cytokine production. The Treg cells exert their immunosuppressive function through dominant consumption of the cytokine interleukin-2 and by inhibitory cytokines (TGF-β, IL-10, IL-35) as well as induction of apoptosis or a killing of effector or antigen-presenting cells (APC) by perforin, granzyme B or Fas ligand interaction. The other immunosuppressive mechanisms of Treg cells involve immune checkpoint molecules and include inhibition of effector T cells by the lymphocyte activation programmed cell death pathways or the cytotoxic T-lymphocyte antigen (CTLA-4). A third immunosuppressive mechanism may be metabolic modulation by indoleamine 2,3-dioxygenase (IDO) expression, which affects the kynurenine-tryptophan pathway in dendritic cells. The Treg cells thereby play a critical role in suppressing autoimmunity and inflammation. Reduced number and function of Treg cells are associated with human autoimmune disease, and activation and augmentation of Treg cells have been shown to be beneficial in treating autoimmune diseases in clinical trials (see the review of Margarita Dominquez-Vallar & David A. Hafler, Regulatory T cells in autoimmune disease, Nature Immunology 2018, 19, 665-673). Overall, all current results suggest that Treg cells contribute to the maintenance of self-tolerance by downregulating the immune response to self and foreign antigens in an antigen-nonspecific manner. Therefore, it is reasonable to hypothesize that a relaxin-2-induced increase in Treg cells in a posttransplant patient also improves post-transplant outcomes beyond the prevention of ischemic injury. The very same can also be assumed when relaxin-2 is used in treating autoimmune induced tissue injury, endothelial cell injury and diseases following endothelial cell activation. The hypothesis is also supported by the fact that a reduction in the proportion of Treg cells in the peripheral blood is known to lift general immune suppression, thereby enhancing the innate and acquired immune response to foreign and self-antigens. Therefore, the present discovery significantly expands the pharmaceutical toolbox.

### EXAMPLES

### Example 1—Relaxin Binds and Activates the Glucocorticoid Receptor

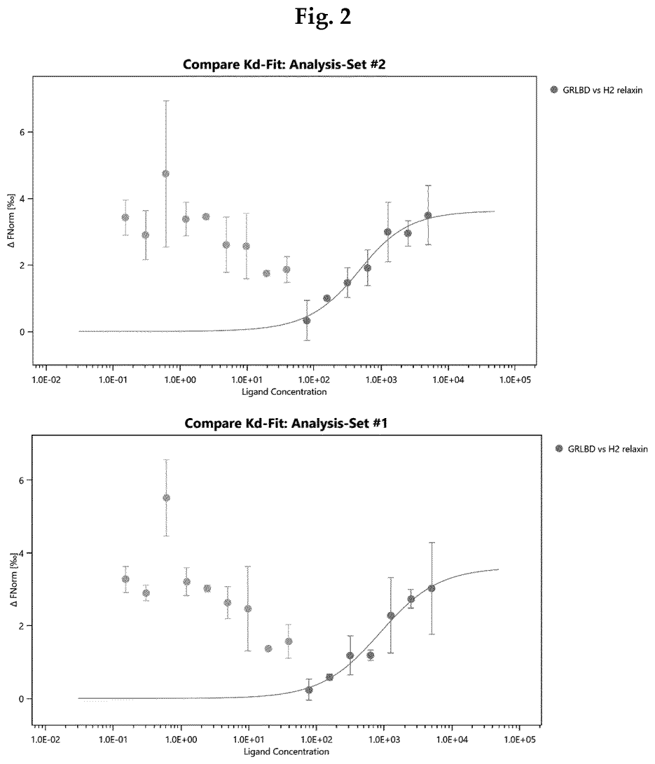

FIG. 1 and FIG. 2 refer to in vitro binding and affinity studies of synthetic human relaxin-2 (shRIx) to the ligand-binding domain of the glucocorticoid receptor (GR-LBD) using microscale thermophoresis (MST). The microscale thermophoresis is based on measuring the directed movement of molecules in localized temperature gradients created by IR laser radiation in high-precision glass capillary tubes containing the interacting partners—synthetic human relaxin-2 and recombinant GR-LBD. For this experiment, the human glucocorticoid receptor (GR-LBD) ligand-binding domain was expressed in an E. coli expression system to obtain a large amount of soluble protein stable for biophysical characterization. The recombinantly produced GR-LBD showed little aggregation and proved to be fully functional. One of the interacting partners was labeled with a fluorescent dye and added to a serial dilution series (15 dilutions each) of the non-fluorescent partner. After incubation, the thermophoretic movement of the complex is detected. Conformational changes due to ligand binding to the target or binding near the fluorophore induce thermophoretic changes. The affinity of interacting protein is determined by analyzing the change in normalized fluorescence as a function of the concentration of titrated binding partner. The next step was to determine the binding mode of relaxin and the activation mechanism of GR. Fluorescence polarisation revealed two binding affinities in the pico-and nanomolar range. Moreover, human H2 relaxin could displace fluormone labeled GS red out of its binding pocket on GR-LBD (see method described by Hemmerling M et al. in Selective Nonsteroidal Glucocorticoid Receptor Modulators for the Inhaled Treatment of Pulmonary Diseases, J. Med. Chem. 2017, 60, 20, 8591-8605). Using this combination of biophysical and structural biology techniques, including microscale thermophoresis (MST), hydrogen-deuterium exchange mass spectrometry (HDX-MS), and NMR, the relaxin-2 binding site of the glucocorticoid receptor was identified, which was the steroid-binding pocket of the GR-LBD.

The effects of relaxin binding on GR-LBD have further been investigated to determine whether relaxin binding activates the receptor like an agonist or represses transcriptional activity by acting like an antagonist. GR-LBD has an activation function-2 site that will recruit cofactors upon ligand binding. The cofactors (coactivators or corepressors) are specific to the cellular environment. Therefore, the binding of relaxin with coactivator and corepressor motifs to the GR-LBD/relaxin complex was tested.

It was found that relaxin binds to both cofactors, resulting in different receptor conformational changes. Since thermophoresis is an intrinsic phenomenon of a molecule that depends on the hydration shell and the size, the binding events could be identified by tracking the associated changes to thermophoresis in a fluorescently labeled interacting partner. In summary, the results of the microscale thermophoresis (see FIGS. 1, and 2) indicate a high-affinity interaction (K_{D}~5 nM) and another lower-affinity interaction (K_{D}~500 nM) of synthetic human relaxin-2 with the GR-LBD.

According to hydrogen-deuterium exchange experiments (not shown), human relaxin-2 appears to bind to helix 12 of the LBD, but in contrast to classical glucocorticoids in the absence of the transcription co-regulator NCoA-2 (nuclear receptor coactivator 2). NCoA-2is also known as glucocorticoid receptor-interacting protein 1 (GRIP1), steroid receptor coactivator-2 (SRC-2), or transcription intermediary factor 2 (TIF2). NCoA-2 contains several nuclear receptor interacting domains and intrinsic histone acetyltransferase activities, and when GR recruits NCoA-2 to a DNA promotion site, the role of NCoA-2 also appears to be in acetylating histones so that the downstream DNA becomes more accessible for transcription. The presence and amount of NCoA-2 are cell-type dependent. NCoA2 (GRIP1, SRC-2, TIF2) therefore supports up-regulation of DNA expression which also leads to increased activation of genes responsible for gluconeogenesis. Since human relaxin-2 does not recruit NCoA-2 upon binding, this type of gene activation does not appear to be triggered when human relaxin-2 binds to the glucocorticoid receptor.

### Example 2—Relaxin-2 Activates Transcription of Genes Dampening Peroxide-Induced Cytotoxicity, Inflammatory Responses, and Apoptosis

With reference to FIG. 3 and FIG. 4, mouse hepatocytes were isolated as described by Tamaki N et al. in Am J Physiol Gastrointest Liver Physiol 2008 294, G499. Briefly, livers from mice anesthetized with pentobarbital were washed and perfused for 5 min with buffer consisting of (all values are mg/L) 8,000 NaCl, 400 KCl, 88,7 NaH₂PO₄·H₂O, 120.45 Na₂HPO₄, 2,380 HEPES, 350 NaHCO₃, 190 EGTA, and 900 glucose, pH 7.25, then treated with 0.03% collagenase at 37 °C for 15 min in digestion buffer containing (all values are mg/L) 8,000 NaCl, 400 KCl, 88.7 NaH₂PO₄·H₂O, 120.45 Na₂HPO₄, 2,380 HEPES, 350 NaHCO₃, and 560 CaCl₂·2H₂O, pH 7.25. After collagenase perfusion, the liver capsule was isolated, and the cells dispersed in Geys balanced salt solution (GBSS)-B consisting of (all values in mg/L) 8,000 NaCl, 370 KCl, 210 MgCl₂·6H₂O, 70 MgSO₄·7H₂O, 120 NaH₂PO₄, 30 KH₂PO₄, 991 glucose, 227 NaHCO₃, and 225 CaCl₂·2H₂O (pH 7.25). Cells were additionally separated by forcing the material through a steel mesh and collecting the cells by centrifugation at 50 G for 1 min. The cell pellet was resuspended in GBSS-B solution and washed three times with intermittent centrifugations.

Isolated mouse hepatocytes were cultured on type I collagen-coated 6-well plates coated at a cell density of 5×10⁵ cells/well with Dulbecco's modified Eagle's medium containing 10% fetal calf serum, 100 U/ml penicillin, and 100 μg/ml streptomycin at 37 °C in a humidified atmosphere of 5% CO 2-95% air. After plating, the medium was replaced with serum-free Dulbecco's modified Eagle medium after 6 hours. The hepatocytes were then subjected to hydrogen peroxide treatment (2 mM H₂O₂/L) for 5 hours with or without pretreatment with synthetic human relaxin-2 (10 nM/L, 24 h) (Relaxera Pharmazeutische GmbH, Bensheim, DE) or dexamethasone (0.5 mM, 24 h) (Sigma Aldrich). In addition, hepatocytes were also transfected with GR-siRNA or scrambled siRNA using lipofectamine reagent (Invitrogen) to test whether the release of LDH or caspase-3 into the medium in these two experiments was dependent on the ligand-activated GR reagent (n =5 for each group). H₂O₂ -induced cell injury (cytotoxicity) was determined immunologically by quantification of lactate dehydrogenase (LDH) released into the culture medium using an enzyme-linked assay according to the manufacturer's instructions (Goat LDH ELISA kit, Biomol Feinchemikalien GmbH, DE) and by Western Blot analysis of cleaved caspase-3 (Caspase-3 Rabbit mAb #14220, Cell Signaling Technology, Danvers, MA, US).

The results are summarized in FIGS. 3 and 4. LDH release data shown in FIG. 3 are expressed as a percentage of detergent-induced maximum cytotoxicity. Data on cleaved/activated caspase-3 shown in FIG. 4 were normalized to β-actin and adjusted for the effect of H₂O₂ (* p<0.05 vs. control; #, p<0.05 vs. H₂O₂; Kruskal-Wallis ANOVA on ranks for global testing with post hoc Mann-Whitney U-tests for pairwise comparisons (Bonferroni-Holm adjustment of p). In summary, FIGS. 3 and 4 show that both relaxin-2 (Rlx) and dexamethasone (Dx) can markedly dampen peroxide-induced cell injury (LDH release) and apoptosis (cleaved caspase-3) and that this attenuating effect does not occur when the expression of GR is specifically knocked out by GR-siRNA, whereas a knock-out using scrambled siRNA (scr-siRNA) showed no effect.

Physiologically, LDH is an enzyme expressed in nearly all living cells, including heart muscles and blood cells, and it catalyzes the conversion of lactate to pyruvate and back. Because it is released during tissue injury, it is a marker of common injuries, damaged tissues, and diseases involving tissue damage such as heart failure. In contrast, the relative concentrations of its substrates primarily regulate LDH activity. LDH is subject to transcriptional regulation by peroxisome proliferator-activated receptor-y coactivator 1α (PGC-1α) in an estrogen-related receptor-α-dependent manner.

) Caspase 3 protein (CASP3) or cysteine-dependent aspartate-directed protease 3 plays an essential role in programmed cell death. Caspase-3 is synthesized as an inactive zymogen until cleaved after apoptotic signaling events. Caspase-3 is thought to ensure that cellular components are degraded in a controlled manner, and that cell death occurs with minimal impact on surrounding tissues. Caspase deficiency has been identified as a cause of tumor development, for example, by a mutation in a cell cycle gene that removes cell growth restrictions in combination with mutations in apoptotic proteins such as caspases that would trigger cell death in abnormally growing cells. Conversely, over-activation of caspase-3 can lead to excessive programmed cell death. This is seen in several neurodegenerative diseases in which neural cells are lost, such as Alzheimer's disease. Caspases involved in processing inflammatory signals are also implicated in disease. Insufficient caspase activation can increase an organism's susceptibility to infection because an appropriate immune response may not be triggered. For example, inflammatory caspase-1 has been linked to the development of autoimmune diseases; drugs that block caspase activation have been used to improve patients' health.

In conclusion, synthetic relaxin-2 as a ligand of the glucocorticoid receptor appears to have cell biological effects like dexamethasone in attenuating an inflammatory response to tissue injury as well as apoptosis and necrosis.

### Example 3—Relaxin-GR Complex Does Not Activate Transcription of Genes Involved in Gluconeogenesis

Mouse hepatocytes were obtained and tested for glucocorticoid-adverse effects after treatment with synthetic human relaxin-2 or dexamethasone (500 nM, 24 hours).

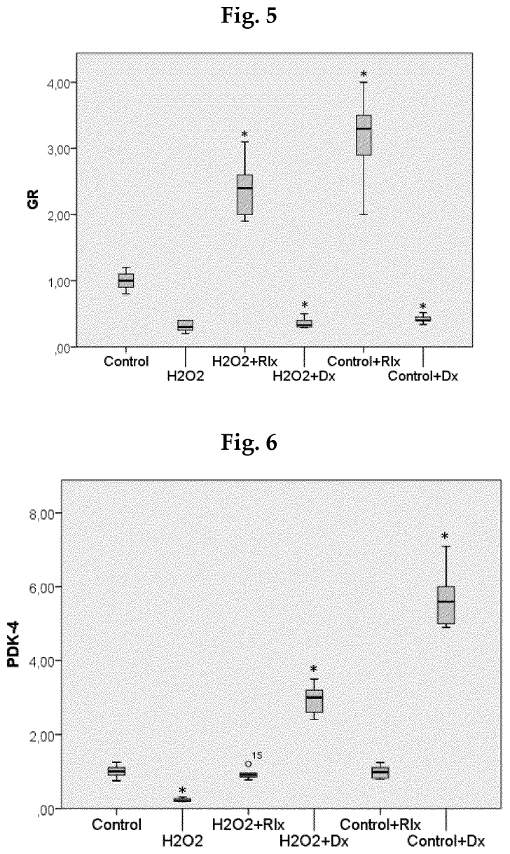

The complex physiological adverse effects of glucocorticoid excess are numerous and difficult to assess (Cushing's syndrome, diabetes, skin thinning, hypertension, osteoporosis, obesity, impaired wound healing, depression, etc.), but measurable in this cell assay is the activation of genes involved in gluconeogenesis and in diabetes, obesity, and impaired wound healing. Consequently, the regulation of GR- and PDK-4 transcription was examined by qRT-PCR in normal primary mouse hepatocytes for control and in H₂O₂ -stressed primary mouse hepatocytes from example 2 after 24 hours of treatment with 10 nM/L synthetic human relaxin-2 (Relaxera Pharma. GmbH&Co. KG) and 500 nM/L dexamethasone (Sigma Aldrich). Results are shown in the block diagrams of FIGS. 5 and 6.

Specifically, FIG. 5 shows that relaxin-2 increases GR gene transcription in normal and H₂O₂ -stressed primary mouse hepatocytes by 100% to 200 %, whereas dexamethasone has no such effect. FIG. 6 shows that the incubation with relaxin-2 has no effect on PDK-4 gene transcription in normal and H₂O₂ -stressed primary mouse hepatocytes. However, when primary mouse hepatocytes are incubated with dexamethasone, PDK-4 gene transcription increases severalfold. The combination of FIGS. 5 and 6 is therefore strong evidence that the complex of relaxin and GR binds to a genomic DNA locus that is different from the DNA locus of steroid-activated GR.

Physiologically, PDK-4 (pyruvate dehydrogenase lipoamide kinase isozyme 4) is a mitochondrial protein that inhibits the pyruvate dehydrogenase complex (PDH) by phosphorylating one of its subunits. An active PDH complex is required to convert pyruvate to acetyl-CoA for the glycolytic products to enter the citric acid cycle. Fasting results in an induction of PDK-4 mRNA and PDK-4 enzyme in both cardiac and skeletal muscle, suppressing glucose oxidation during starvation as part of an integrative response and for glucose maintenance. The PDK-4 enzyme is therefore thought to play a critical role in regulating glucose metabolism, whereas an increased PDK-4 transcription indicates gluconeogenesis. It is well known that PDK-4 expression is physiologically regulated by glucocorticoids, retinoic acid, and insulin which enhance the transcription of the PDK-4 gene in white adipose tissue. Oxidation of fatty acids is also increased when the PDK-4 level is elevated. Insulin downregulates PDK-4 mRNA transcription. When cells are exposed to dexamethasone to increase PDK-4 mRNA expression, insulin blocks this effect and the oxidation of fatty acids. In type 2 diabetes, PDK-4 is overexpressed in skeletal muscle, resulting in impaired glucose utilization. In patients suffering from obesity, PDK-4 mRNA expression is also markedly decreased in association with increased glucose uptake, likely due to the downregulation of PDK-4 by insulin. This is consistent with the hypothesis that fatty acid availability affects glucose metabolism by regulating the pyruvate dehydrogenase (PDH) complex. Indeed, in insulin-resistant individuals, an inadequate downregulation of PDK-4 mRNA may cause increased PDK-4 expression, leading to impaired glucose oxidation followed by increased fatty acid oxidation. Conversely, PDK-4 is downregulated in cardiac muscle tissue during heart failure, which is a physiological countermeasure (Razeghi P et al., in Downregulation of metabolic gene expression in failing human heart before and after mechanical unloading, Cardiology 2002, 97(4): 203-9).

The results in FIGS. 3 through 6 show that relaxin-2 like glucocorticoids, corticosteroids, and mineralocorticoids has anti-inflammatory effects, but without leading to impaired glucose oxidation, increased fatty acid oxidation, and gluconeogenesis. The ubiquitous role of PDK-4 further suggests that a pharmaceutical composition containing relaxin-2 and treatment with relaxin-2 may be an alternative to glucocorticoid treatment because it results in increased expression of the glucocorticoid receptor, which suppresses immune and inflammatory responses, but not in increased expression of PDK-4, which is unfavorable for glucose metabolism and balance.

The glucocorticoid receptor (GR) is an evolutionarily conserved ligand-dependent transcription factor. Upon binding of a steroid hormone or other ligand, the receptor migrates from the cytoplasm to the nucleus where it binds to a genomic DNA locus and positively or negatively modulates the transcription rates of the locus-associated genes. Tremendous efforts have been made to uncover the molecular signaling actions of the GR, including intracellular shuttling, transcriptional regulation, and interaction with other intracellular signaling pathways. In brief, glucocorticoids are essential for the maintenance of resting-state and stress response and, therefore, they are essential in the treatment of numerous diseases, including autoimmune, inflammatory, allergic, and lymphoproliferative disorders. The pathological or therapeutic implications of the GR cannot be overstated. These include, except for genetic alterations in the human GR gene, disease-associated GR regulatory molecules and the development of GR ligands with selective GR action.

FIGS. 1 to 6 show that the administration of synthetic relaxin-2 has therapeutic effects that differ from known corticosteroids and glucocorticoids. The experiments presented demonstrate that synthetic relaxin-2 elicits GR-dependent glucocorticoid effects, including inhibition of apoptosis in mouse hepatocytes and inhibition of cytokine release in human macrophages (see example 4 below), which, in contrast to classical corticosteroids and glucocorticoids, avoid unwanted effects such as GR down-regulation or glucocorticoid- or steroid-induced hyperglycemia or activation of gluconeogenesis.

### Example 4-Relaxin-2 Dampens the Release of Pro-inflammatory Cytokines

THP-1 cells were differentiated into macrophages and cultured as described by Dschietzig T et al. in Identification of the pregnancy hormone relaxin as a glucocorticoid receptor agonist, FASEB J 2004, 18:1536-1538. Briefly, the THP-1 cells are derived from a cell line generated from a human monocyte leukemia, and when the cells are treated in the passage with myristate-phorbol ester, they differentiate into macrophages as is well known in the art.

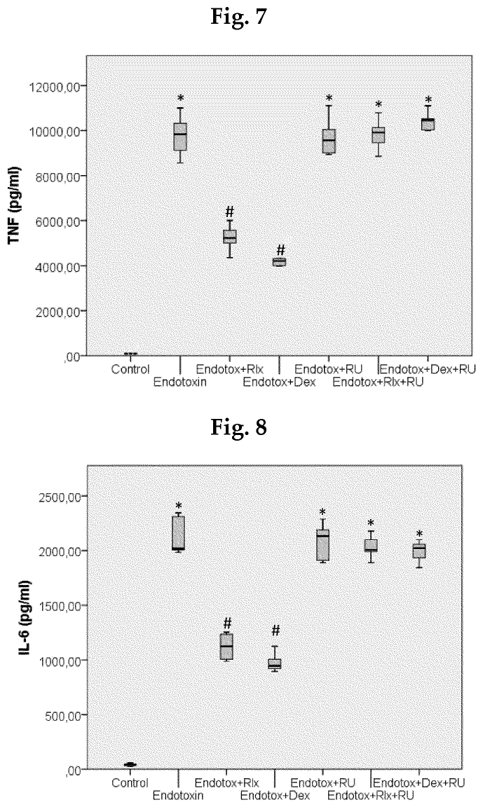

Referring to FIGS. 7 and 8, the macrophages were then challenged for 24 hours with 10 ng/ml Salmonella abortus equii endotoxin (Sigma Aldrich) in the presence or absence of synthetic human relaxin-2 (10 nM/L) and dexamethasone (500 nM(L) and/or the GR antagonist RU-486 (500 nM) (Sigma Aldrich), or combinations thereof (n=5 for each group). RU-486 binds to the GR in the steroid pocket of the ligand-binding domain. Subsequently, the supernatant levels of TNF-α and interleukin-6 were determined by ELISA (R&D Systems).

The results are summarized in the block diagrams for FIGS. 7+8, and demonstrate that the endotoxin induced these macrophages to produce and excrete the proinflammatory cytokines tumor necrose factor alpha (TNF) and interleukin-6 (IL-6), which inflammatory response was both depressed in the presence of synthetic human relaxin-2 and dexamethasone. This response can be classified as GR-dependent, as RU-486 completely inhibited this effect.

As further background, RU-486, also known as Mifepristone®, is a steroidal antiprogesterone as well as an antiglucocorticoid and antiandrogen. It competitively antagonizes cortisol action at the GR receptor. In humans, an antiglucocorticoid effect of RU-486 is observed at doses greater or equal to 4.5 mg/kg through a compensatory increase in adrenocorticotropic hormone (ACTH) and cortisol. In animals, a weak antiandrogenic effect is seen with prolonged administration of very high doses (Danco Laboratories, 2005, Mifeprex U.S. prescribing information). Thus, this example demonstrates that the administration of synthetic human relaxin-2 can provide GR-mediated regulation of the immune system, and has immune-suppressive properties, but without the side effects of inducing gluconeogenesis and insulin insensitivity.

### Example 5—Relaxin-GR Complex Does Not Induce Hyperglycemia in Contrast to the Dexamethasone-GR Complex

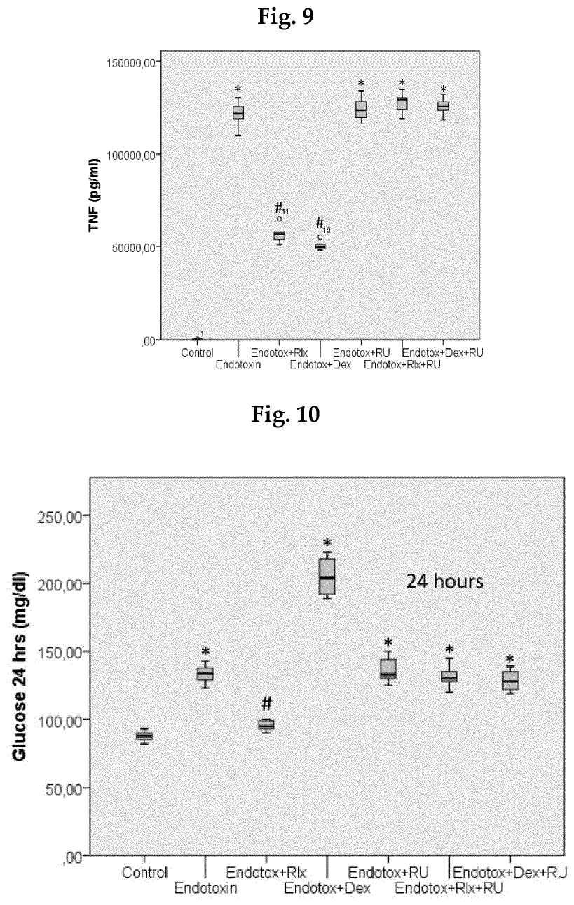

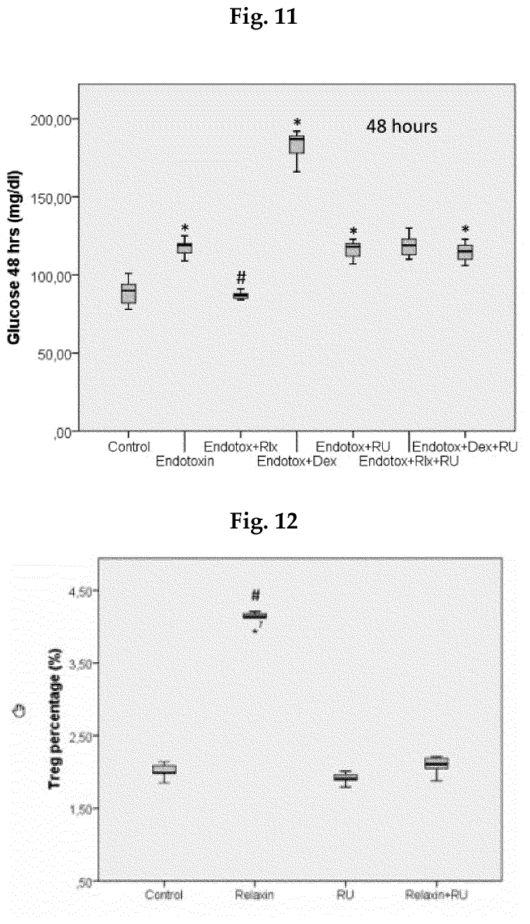

FIGS. 9, 10, and 11 refer to animal experiments and show the different effects of relaxin-2 and dexamethasone (Sigma Aldrich) administration on blood glucose levels in rats after 24 and 48 hours. In brief, male and female Sprague-Dawley rats (body weight 300-350 g) were treated with an intraperitoneal injection of E. coli endotoxin (125 μg/kg body weight) or placebo (vehicle). Blood from the tail vein was taken after 24 hours for the determination of circulating TNF-α (ELISA, R&D Systems) and fasting blood glucose and at 48 hours for measurement of fasting blood glucose. Two hours before administration of endotoxin or placebo, animals received dexamethasone (intramuscular 10 mg/kg body weight), synthetic relaxin-2 (Relaxera Pharmazeutische GmbH&Co. KG, Bensheim) as a subcutaneous infusion (4 μg shRIx/h) over 12 hours via osmotic Alzet minipumps, oral RU-486 (single dose of 10 mg/kg body weight), or combinations thereof (n=5 animals per group). The results are shown in FIGS. 10 and 11; *, p<0.05 vs. control; #, p<0.05 vs. endotoxin plus dexamethasone; Kruskal-Wallis ANOVA on ranks for global testing with posthoc Mann-Whitney U-tests for pairwise comparisons (Bonferroni-Holm adjustment of p).

While dexamethasone markedly enhanced the endotoxin-related increase in blood glucose, reflecting the animal's disease and hyperthermia response to endotoxin, relaxin-2 decreased glucose levels compared with endotoxin alone. This was true at both 24 and 48 hours after endotoxin. Oral administration of RU-486 inhibited the effects of relaxin-2 and dexamethasone by its antagonistic actions at the glucocorticoid receptor, as also the concentration of TNF-α in the circulation. In conclusion, human relaxin-2 attenuated the endotoxin-induced surge of circulating TNF-α in rats of both sexes. The effect could be identified as GR-dependent and comparable to that of dexamethasone, a classic glucocorticoid. However, unlike dexamethasone, relaxin-2 did not induce hyperglycemia which is a medically extremely important finding.

### EXAMPLE 5-Relaxin—2 furthers Differentiation of naïve T Cells into Regulatory T Cells (Treg) in Mice Similar to Glucocorticoids

Mice of C57BI/6 background (each groups n=5) were intraperitoneally injected once daily for 3 consecutive days in the following experimental groups: synthetic human relaxin-2 (Relaxera) (10 micrograms/kg body weight), placebo (vehicle, sodium acetate), RU-486 (2.5 mg/kg body weight), relaxin-2 plus RU-486. Thereafter, mice were sacrificed, their spleens were processed using standard procedures, and the percentage of spleen regulatory T cells (T_{reg}) was analyzed by FACS. T_{reg} cells were originally defined as characterized as CD4+FoxP3+ cells wherein FoxP3 (a Forkhead transcription factor) is the T_{reg} master regulator. Regulatory T cells expressing the transcription factor Forkhead box P3 (FoxP3) are known to control immune responses and prevent autoimmunity.

(058) As shown in FIG. 12 the administration of relaxin-2 approximately doubled the percentage of regulatory T cells (CD4+FoxP3+ cells) as compared with placebo. The GR antagonist RU-486 did not affect the differentiation of T cell but when administered together with shRIx, its presence completely abrogated the relaxin-induced T_{reg} increase. #, p<0.05 vs. control; Kruskal-Wallis ANOVA on ranks for global testing with posthoc Mann-Whitney U-tests for pairwise comparisons (Bonferroni-Holm adjustment of p).

These findings, therefore, substantiate a stimulatory effect of synthetic human relaxin-2 on the differentiation of peripheral (spleen) regulatory T cells (T_{reg}) in mice and show that these effects are GR-dependent because they could be nullified by a GR-antagonist such as RU-486.

### Discussion and Synopsis

As mentioned above, glucocorticoids and corticosteroids are mainstays in the treatment of tissue-damaging autoimmune pathologies such as rheumatoid arthritis, and they are immunosuppressants following organ transplantation. An overwhelming amount of literature exists on the glucocorticoid-mediated regulation of the immune system and in particular on the glucocorticoid-mediated regulation of innate immunity and inflammation. The calcium-binding S100 proteins are universal markers for inflammation and of the innate immune system, notably calprotectin (the complex of S100A8 and S100A9) as well as S100A12. Generally, the presence of S100A12 and calprotectin indicate tissue injury, endothelial cell activation, and inflammation-mediated responses. Cell stress and/or inflammation induce the release of S100 proteins to acellular compartments where they bind cell surface receptors such as RAGE, TLR4, CD147, and GPCR. The interactions between the calcium-binding S100 proteins and their receptors activate intracellular signaling pathways such as AP1 and NFKB, which further initiates multiple cellular processes such as cell differentiation, migration, apoptosis, proliferation, and inflammation; activator protein 1 (AP1), extracellular signal-regulated protein kinase (ERK), G-protein-coupled receptor (GPCR); interleukin 1 (IL-1); interleukin 7 (IL-7), nuclear factor of kappa light polypeptide gene enhancer in B-cells inhibitor alpha (IKBa), c-Jun N-terminal kinase (JNK), p38 mitogen-activated protein kinase (P38), the receptor for advanced glycation end products (RAGE), toll-like receptor 4 (TLR4), TNF receptor-associated factor 2 (Traf2). Mammalian cells secrete calprotectin during the inflammatory response. The exact mechanism by which the complex of S100A8/S100A9 is secreted by mammalian cells during inflammation remains unknown. When released to the extracellular space, S100 proteins have activities in the regulation of immune homeostasis, post-traumatic injury, tissue damage, and inflammation. S100 proteins trigger inflammation through interacting with receptors RAGE and TLR4, and there is evidence that calprotectin (S100A8/S100A9) is an endogenous agonist of TLR4. Binding to TLR4 initiates a signaling cascade and regulates inflammation, cell proliferation, and differentiation in an NFKB-dependent manner. Apart from TLR4, RAGE has also been suggested to bind S100 proteins such as S100A8, S100A12, S100A8/A9 (calprotectin), and S100B. By interacting with RAGE, S100 proteins activate NFKB, inducing the production of pro-inflammatory cytokines leading to the migration of neutrophils, monocytes, and macrophages. Extracellular S100 proteins are therefore involved in the regulation of cell apoptosis, migration of monocytes, macrophages, neutrophils, lymphocytes, myoblasts, epithelial cells, and endothelial cells. Consequently, the levels of S100A8/A9 complex (calprotectin) and S100A12 in extracellular fluids can be used as biomarkers to assess the degree of inflammatory regulation and tissue injury.

Liver transplantation and cold storage of livers were studied in mice having the same genetic setup. In this syngeneic mouse model, all immunological responses due to surgical trauma, cold storage, and ischemia injury are therefore mediated by the innate immune system whose effects can be dampened by the administration of corticosteroids. In this syngeneic mouse model, the administration of relaxin-2 at cold storage and/or at reperfusion proved cell-protective and markedly improved posttransplant liver function and survival (cf. Kageyama S et al. in Recombinant relaxin protects liver transplants from ischemia damage by hepatocyte glucocorticoid receptor: From bench-to-bedside, Hepatology 2018, 258-273). Although the authors hypothesize a regulatory role of the relaxin-2-GR complex in the inflammatory injury in liver transplants, the impact of relaxin on the promotion of peripheral Treg cells and the activation and promotion of immune-suppressive Treg cells was not noted. With the knowledge of the present invention, continuous treatment of the recipient with relaxin-2 is warranted as the activated peripheral Treg cells can suppress local immune responses and responses activated by any kind of tissue-and endothelial injury. A continuous administration of relaxin-2 is also warranted as relaxin-2 does not lead to gluconeogenesis as shown in FIGS. 10 and 11.

Concerning the anti-cancer activity of glucocorticoids, for example, De Bono et al. 2014 (Clin Cancer Res 2014, 20:1925-1934, US2006018910) disclose treating hormone-refractory prostate cancer patients with a combination of docetaxel, anti-IGF-IR antibodies, and dexamethasone, since the glucocorticoid receptor is upregulated in refractory prostate cancer cells, and therefore should be inhibited to impair the proliferation of these cancer cells (Ruhr et al., 2018. Clin Cancer Res 24:927-938). It was found that activation of the glucocorticoid receptor leads to the acquisition of quiescence, subserved by cell cycle arrest through p57 and reprogramming of signaling orchestrated via Insulin Receptor Substrate 2 (IRS2)/Forkhead Box 01 (FOXOI). As synthetic relaxin-2 can be used as a substitute for dexamethasone, it may be favorably used in such therapy without incurring the adverse effects of glucocorticoids and their analogs.

Boehnert MU in Relaxin as an additional protective model of isolate perfused rat liver, Ann N Y Acad Sci 2005, 1041:434-440 and Bausys A et al. in Custodio/® supplemented with synthetic human relaxin decreases ischemia-reperfusion injury after porcine kidney transplantation, Int J Mol Sci. 2021, 22, 11417 describe that relaxin in the perfusion solution reduces ischemia-reperfusion injury (IRI) after kidney or liver transplantation, and that relaxin-2 (RLX) upregulates the expressions of mitochondrial superoxide dismutase-2 (SOD2) and nuclear factor kappa B (NFKB), a regulator of innate immunity. The expression of receptor-interacting serine/threonine-protein kinase 1 (RIPK1), which plays a role in apoptosis and necroptosis, was downregulated compared to controls. Also downregulated was the expression of mixed lineage kinase domain-like protein (MLKL), which plays a role in tumor necrosis factor (TNF)-induced necroptosis, and the number of caspase 3- and MPO-positive cells was also decreased in the grafts after static cold storage in a solution containing relaxin-2. Static cold storage in a cardioplegic solution is the simplest, most convenient, and least expensive method of organ preservation in clinical practice. While relaxin-2 has been described for its antifibrotic, antioxidant, anti-inflammatory, and cytoprotective properties, there was no evidence that relaxin-2 can be used beneficially as a substitute for glucocorticoids without inducing their cushingoid adverse effects, particularly gluconeogenesis, and its promotion of immune suppressive Treg cells was also not observed, which, however, makes relaxin-2 a broad-spectrum drug for tissue and endothelial injury, rather than a vasodilator additive in a perfusion solution to prevent ischemia-reperfusion injury.

1.-15. (canceled)

16. A method of treating a patient in need of prolonged dampening of inflammatory responses caused by or associated with an activated innate immune system wherein side effects associated with steroid treatment are to be avoided, the method comprising
(a) formulating synthetic human relaxin-2 in a composition for subcutaneous injection, comprising a pharmacological solvent, diluent, or excipient and configured for continuous or prolonged administration, and
(b) administering to the patient an effective amount of synthetic human relaxin-2.

17. A method of treating a patient as claimed in claim 16 wherein the patient is in need of long-term suppression of inflammatory responses caused by or associated with an activated innate immune system and wherein side effects associated with glucocorticoid, corticosteroid, mineralocorticoid, or other steroid therapy are to be avoided, the method comprising
(a) formulating synthetic human relaxin-2 in a composition for subcutaneous injection, comprising a pharmacological acceptable solvent, diluent, or excipient and configured for continuous or prolonged administration, and
(b) administering to the patient an effective amount of synthetic human relaxin-2, wherein administration thereof does not provoke a manifestation or deregulation of diabetes, wound-healing disorder, and/or the symptoms of Cushing's syndrome.

18. A method of treating a patient as claimed in claim 16 wherein the patient, or an organ thereof, or an allotransplant is affected by immune reactions that cause tissue or endothelium damage, and who is having one or more of the following clinical parameters elevated relative to those observed in healthy individuals or organs:, the elevated clinical parameters comprising:
a) an elevated serum HMGB1 (high-mobility group box protein);
b) an elevated serum sTLR4 (soluble Toll-like receptor-4),
c) an elevated serum sRAGE (soluble receptor of advanced glycation end-products);
d) an elevated serum calprotectin and/or serum S100A12 or elevated calprotectin and/or S100A12 in the organ's post-donation flush.

19. A method of treating a patient as claimed in claim 16 wherein the patient is suffering from an inflammatory or immune-mediated disorder selected from systemic inflammatory response syndrome (SIRS), autoimmune or rheumatic diseases, thyroiditis, gastritis, insulitis, sialoadenitis, adrenalitis, oophoritis, glomerulonephritis, polyarthritis, ankylosing spondylitis (AS) and spondylarthrites, fibromyalgia, gout, infectious arthritis, lupus, systemic autoimmune disease, osteoarthritis (OA), psoriatic arthritis (PsA) and inflammatory types of arthritis, rheumatoid arthritis (RA), SARS-Covid 19 and severe acute respiratory syndrome (SARS).

20. A method of treating a patient as claimed in claim 16, wherein the patient displays the following clinical criteria: pre-diabetes (HbA1C>5.7 and <6.5 %), obesity (BMI>30 kg/m2), hypertension (stage 1 or higher according to the 2017 ACC/AHA Guidelines).

21. A method of treating an allograft recipient as claimed in claim 18 wherein one or more of the following four conditions is satisfied:
(a) serum HMGB 1 (high-mobility group box protein) concentration of at least 2 ng/mL; (b) a serum sTLR4 (soluble Toll-like receptor-4) concentration of at least 0.25 ng/ml; (c) a serum sRAGE (soluble receptor of advanced glycation end-products) of at least 0.5 ng/ml, and/or
d) a serum calprotectin of at least 4 micrograms/mL.

22. A method of treating an allograft recipient as claimed in claim 18 who has been subjected to increased immunological response reactions by the innate immune system and when one or more of the following criteria are observed in the blood flushed out after the organ donation:
(a) serum HMGB 1 (high-mobility group box protein) concentration of at least 2 ng/ml; (b) a serum sTLR4 (soluble Toll-like receptor-4) concentration of at least 0.25 ng/ml; (c) a serum sRAGE (soluble receptor of advanced glycation end-products) of at least 0.5 ng/ml, or
d) a serum calprotectin of at least 4 micrograms/mL.

23. A method of treating a patient as claimed in claim 16, wherein the patient is need of hormone-refractory cancer therapy, the cancer being selected from the group consisting of:
(i) prostate cancer;
(ii) breast cancer;
(iii) cancers treatable by a primary cancer therapy mediated by a ligand-activated glucocorticoid receptor (GR) pathway, including, but not limited to, multiple myeloma, Hodgkin's disease, and other lymphoid cancers; and
(iv) Kaposi sarcoma,
wherein synthetic human relaxin-2 is administered as a supplement to or substitute for a glucocorticoid, cortisone, glucocorticoid receptor activating hormone or other steroid.

24. A method of treating a patient, the method comprising:
(a) testing the patient for one or more clinical parameters selected from the group consisting of:
(i) serum HMGB1 (high-mobility group box protein) at a concentration of at least 4 ng/ml;
(ii) serum sTLR4 (soluble Toll-like receptor-4) at a concentration of at least 0.5 ng/ml;
(iii) serum sRAGE (soluble receptor of advanced glycation end-products) at a concentration of at least 2 ng/ml; and
(iv) serum calprotectin at a concentration of at least 10 μg/ml; and
(b) administering to the patient an effective amount of synthetic human relaxin-2 in a composition comprising a pharmacologically acceptable solvent, diluent, or excipient, wherein administration thereof dampens or suppresses physiologic inflammatory responses mediated by the innate immune system.

25. A method of treating a patient as claimed in claim 24, wherein the patient is suffering from an inflammatory or immune-mediated disorder selected from systemic inflammatory response syndrome (SIRS), autoimmune or rheumatic diseases, thyroiditis, gastritis, insulitis, sialoadenitis, adrenalitis, oophoritis, glomerulonephritis, polyarthritis, ankylosing spondylitis (AS) and spondylarthrites, fibromyalgia, gout, infectious arthritis, lupus, systemic autoimmune disease, osteoarthritis (OA), psoriatic arthritis (PsA) and inflammatory types of arthritis, rheumatoid arthritis (RA), Covid 19 and severe acute respiratory syndrome (SARS).

26. A method of treating a patient as claimed in claim 24, wherein the patient is suffering from immunological reactions mediated by the innate immune system and/or exhibits one or more of the following clinical parameters: (i) pre-diabetes, defined as a haemoglobin A1C (HbA1C) level greater than 5.7% and less than 6.5%; (ii) obesity, defined as a body mass index (BMI) greater than 30 kg/m2; (iii) hypertension, defined as stage 1 or higher according to the 2017 American College of Cardiology/American Heart Association (ACC/AHA) Guidelines.

27. A method of treating a patient as claimed in claim 24, wherein the patient is suffering from inflammatory responses mediated by the innate immune system and/or requires suppression of inflammatory responses via activation of a ligand-activated glucocorticoid receptor, the method comprising administering an effective amount of synthetic human relaxin-2, wherein administration of the synthetic human relaxin-2 avoids or mitigates the manifestation or deregulation of diabetes, wound-healing disturbances, and/or the symptoms of Cushing's syndrome.

28. A method of treating a patient as claimed in claim 24, wherein the patient has received an allograft and requires suppression of the innate immune system and inflammatory responses, after testing the patient positive on one or more of the following clinical conditions:
(i) serum HMGB1 (high-mobility group box protein) concentration greater or equal to 2 ng/ml,
(ii) serum sTLR4 (soluble Toll-like receptor-4) concentration greater or equal to 0.25 ng/ml;
(iii) serum sRAGE (soluble receptor of advanced glycation end-products) concentration greater or equal to 0.5 ng/ml; and/or (iv) serum calprotectin concentration greater or equal to 4 micrograms/mL.

29. A method of treating a patient as claimed in claim 24, wherein the patient is in need of hormone-refractory cancer therapy, the cancer being selected from prostate cancer, breast cancer, and cancers treatable via a primary cancer therapy mediated by a ligand-activated glucocorticoid receptor, including, but not limited to, multiple myeloma, Hodgkin's disease, and other lymphoid cancers, and Kaposi's sarcoma, wherein an effective amount of synthetic human relaxin-2 is administered as a supplement to or substitute for a glucocorticoid receptor-activating hormone.

30. A method of treating a patient diagnosed as requiring immunosuppressive therapy, the method comprising:
(a) administering an effective amount of synthetic relaxin-2 as a supplement to or substitute for a glucocorticoid receptor-activating hormone, wherein such administration prevents or avoids a manifestation or deregulation of diabetes and/or symptoms of Cushing's syndrome, and
(b) wherein the patient exhibits one or more of the following clinical features: (i) pre-diabetes, defined as a haemoglobin A1C (HbA1C) level greater than 5.7% and less than 6.5%); (ii) obesity, defined as a body mass index (BMI) greater than 30 kg/m2; and (iii) hypertension, defined as Level 1 or higher according to the 2017 American College of Cardiology/American Heart Association (ACC/AHA) Guidelines.
